(19) United States
(12) Patent Application Publication
(10) Pub. No.: US 2025/0230503 A1
(43) Pub. Date: Jul. 17, 2025

(54) METHODS AND COMPOSITIONS RELATING TO PERSONALIZED PAIN MANAGEMENT

(71) Applicant: Children's Hospital Medical Center, Cincinnati, OH (US)

(72) Inventors: Vidya Chidambaran, Cincinnati, OH (US); Hong Ji, Cincinnati, OH (US); Lisa J. Martin, Cincinnati, OH (US)

(73) Assignee: Children's Hospital Medical Center, Cincinnati, OH (US)

(21) Appl. No.: 18/853,768

(22) PCT Filed: Mar. 31, 2023
(86) PCT No.: PCT/US2023/017011

§ 371 Date: Oct. 3, 2024

Related U.S. Application Data
(60) Provisional application No. 63/327,776, filed on Apr. 5, 2022.

(51) Int. Cl. C12Q 1/6883; A61K 31/245; A61K 31/706; A61K 31/7068

(52) U.S. Cl. CPC C12Q 1/6883; A61K 31/245; A61K 31/706; A61K 31/7068; C12Q 2600/118; C12Q 2600/154

(57) ABSTRACT

The disclosure relates to methods for pain management in the perioperative context, particularly through the use of one or more biomarkers such as the DNA methylation status in genes of the PARK16 locus.

### STATEMENT REGARDING FEDERALLY-SPONSORED RESEARCH

This invention was made with U.S. Government support under 5K23HD082782, awarded by the Eunice Kennedy Shriver National Institute of Child Health and Human Development of the National Institutes of Health, R21 HG008186 and R01 NS099068 awarded by the National Institutes of Health and under R21 AI119236 awarded by the National Institutes of Health and the National Institute of Allergy and Infectious Disease. The U.S. Government has certain rights in the invention.

### TECHNICAL FIELD

The present invention relates to the field of medicine, and more particularly to the field of pain and anxiety management, especially in the context of post-surgical pain.

### BACKGROUND

Chronic postsurgical pain (CPSP) is often defined as pain that lasts beyond three months post-surgery, in the absence of other preexisting problems or postoperative complications (Macrae W A Brit. J. Anaesthesia 2008; 101(1):77-8). In children, the median prevalence of CPSP is 20% (Rabbitts J A et al., J. of Pain 2017; 18(6):605-614), however the incidence ranges from 11-54% after spine fusion (Chidambaran V et al., Eur. J. of Pain 2017; 21(7):1252-1265; Landman Z et al., Spine 2011; 36(10):825-829; Sieberg C B et al., J. of Pain 2013; 14(12):1694-1702) a painful surgery that adolescents undergo. CPSP is a classic example of gene-environment interaction, and involves multiple peripheral and central signaling and modulatory pathways regulated by genes (James S K Brit. J. of Pain 11(4):178-185). It has a heritable risk of 45%, (Young E E et al., J. Med. Genet. 2012; 49(1):1-9) and genetic factors explain some of the individual differences in pain perception (Angst M S et al., Pain 153(7):1397-1409; Norburv T A et al., Brain 2007; 130(11):3014-3049). However, a genetic basis for CPSP has been elusive (Katz J. Pain 2012; 153(3):505-506) attributed partly to lack of replicability (Kim H et al., J. Pain 10(7):663-693) and inconsistent findings (Sadhasivam S et al., Pharmacogenomics 2012; 13(15):1719-1740) in genetic association studies (Branford R et al., Clin. Genet. 2012; 82(4):301-310; Walter C et al., Pain 2009; 146(3):270-275) and lack of consideration of gene-environmental interactions. Especially in children, caregiving environment and psychological factors like anxiety, prime children's pain responses, influence the ‘epigenetic landscape’ and influence his or her response to further surgical stress (Denk F et al., Neuron 2012; 73(3):435-444; Hettema J M et al., Arch. Gen. Psychiatry 2005; 62(2):182-189). Twin studies have shown that environmental factors are involved in the inter-personal differences in pain sensitivity (Angst M S et al., Pain 153(7):1397-1409). Elucidating gene-environmental influences through epigenetics is expected to explain critical gaps in predisposition and mechanisms involved in CPSP (Crow M et al., Genome Med. 2013; 5(2):12-21; Doerhing A et al., Eur. J. Pain 2011; 15(1):11-16).

DNA methylation via addition of a methyl group to the 5′ position of a cytosine-guanine residue (CpG dinucleotide) is a common epigenetic mechanism associated with decreased transcriptional activity, and altered expression of nociceptive genes. It affects pain processing and the transition from acute to chronic pain (Bucheit T et al., Pain Medicine 2012; 13(11):1474-1490). Psychological, perioperative and μ-opioid receptor gene (OPRM1) DNA methylation markers were recently identified as predictors of acute and chronic postsurgical pain in adolescents undergoing spine fusion surgery. The OPRM1 DNA methylation levels have been found to be elevated in opioid and heroin addicts (Chorbov V M et al., J. Opioid Manag. 2011; 7(4):258-264). Levenson et al., (U.S. Ser. No. 12/631,622) developed a DNA methylation-based test for detecting and monitoring methylation states of biomarker genes which differ in the diseased compared to the non-diseased state (e.g., multiple sclerosis and breast cancer). However, effect sizes of single CpG sites are small, and sometimes identify associations that cannot be replicated. Hence, this study uses epigenome-wide association studies (EWAS) and a global bioinformatics-based approach to identify pathways, histone marks, and protein-DNA binding events enriched in DNA methylation differences associated with CPSP and anxiety. This approach integrates epigenetic-level data with biologic processes, pathways, and networks, and overcomes pitfalls of hypothesis-driven candidate marker association studies (Zorina-Lichenwalter K et al., Neuroscience 2016; 338:36-62). EWAS also allows novel candidate discovery, and have previously been used to study epigenetics of other conditions (e.g. panic disorder) (Shimada-Sugimoto M et al., Clinical Epigenetics 2017; 9(1):6-16) but not CPSP. It will test the hypothesis that shared biological processes enriched in DNA methylation will be associated with CPSP and anxiety, which will suggest new avenues for preventing and treating CPSP.

Therefore, there is a need to identify clinical markers for predicting a patient's susceptibility to CPSP in order to provide improved management of pain in the clinical setting.

### SUMMARY

This work follows a previous epigenome-wide study in children undergoing spine fusion surgery showing that DNA methylation at CpG sites in genes enriching opioid, dopaminergic and GABA pathways was associated with CPSP and a systems biology guided study delineating genetic pathways involved in the pathophysiology of CPSP. The present invention is based upon integration of DNA methylation analyses with genetic studies in order to elucidate the mediating molecular mechanisms underlying CPSP. The present inventors discovered CPSP-associated methylation quantitative trait loci (meQTL) at the PARK16 locus using blood samples of patients with idiopathic scoliosis undergoing posterior spine fusion using standard surgical techniques, anesthetic and pain protocols. MeQTLs are genetic polymorphisms (such as single nucleotide polymorphisms, or SNPs) that influence the level of DNA methylation at proximal CpG sites. In accordance with the present invention, the meQTLs rs960603 (PM20D1) and rs708727 (RAB7L1) are identified as associated with CPSP. In the context of the present invention “the PARK16 locus” refers to a genetic region spanning five genes on chromosome 1q32 of the human genome. The five genes that make up the PARK16 locus include SLC45A3, NUCKS1, RAB7, RAB7L1, SLC41A1, and PM20D1. At this locus, CPSP risk meQTLs were associated with decreased DNA-methylation at RAB7L1 CpG sites (referred to as “hypomethylation”) and increased DNA-methylation at PM20D1 CpG sites (referred to as “hypermethylation”). Corresponding RAB7L1/PM20D1 blood eQTLs (GTEx) and cytosine-guanine dinucleotide-loci enrichment for histone marks, transcription factor binding sites and ATAC-seq peaks indicate a mechanism involving altered transcription factor-binding.

In embodiments, the disclosure provides a method for the prophylaxis or treatment of perioperative pain in a patient in need thereof, the method comprising assaying, in vitro, a biological sample from the patient to determine the DNA methylation status of at least one CpG site at the PARK16 locus. In embodiments, the at least one CpG site is in a north or south shore of a CpG island on RAB7L1 and/or PM20D1. In embodiments, a DNA methylation status of hypomethylation on RAB7L1 CpG sites indicates the subject is at high risk of CPSP. In embodiments, a DNA methylation status of hypermethylation on PM20D1 CpG sites indicates the subject is at high risk of CPSP. In embodiments, a DNA methylation status of hypomethylation on RAB7L1 CpG sites and hypermethylation on PM20D1 CpG sites indicates the subject is at high risk of CPSP.

In embodiments, the disclosure provides a method for identifying a patient who is susceptible to perioperative pain, the method comprising assaying, in vitro, a biological sample from the patient to determine the DNA methylation status of at least one CpG site at the PARK16 locus. In embodiments, the patient is determined to be at risk of CPSP where the method determines decreased DNA methylation at RAB7L1 CpG sites and increased DNA methylation at PM20D1 CpG sites.

In embodiments of the methods described here, hypomethylation of one or more of the RAB7L1 CpG sites designated cg16031515 and cg16031515 in Table 1 identifies the patient as susceptible to perioperative pain. In embodiments of the methods described here, hypermethylation of one or more of the PM20D1 CpG sites designated cg14893161, cg12898220, cg11965913, cg07167872, cg16334093, and cg07157834 in Table 1 identifies the patient as susceptible to perioperative pain. In embodiments, the patient identified as susceptible to perioperative pain is one in which the RAB7L1 CpG sites designated cg16031515 and cg16031515 are determined to be hypomethylated in a biological sample obtained from the patient. In embodiments, the patient identified as susceptible to perioperative pain is one in which the PM20D1 CpG sites designated cg14893161, cg12898220, cg11965913, cg07167872, cg16334093, and cg07157834 are determined to be hypermethylated in a biological sample obtained from the patient. In embodiments, the patient identified as susceptible to perioperative pain is one in which the RAB7L1 CpG sites designated cg16031515 and cg16031515 are determined to be hypomethylated and the PM20D1 CpG sites designated cg14893161, cg12898220, cg11965913, cg07167872, cg16334093, and cg07157834 are determined to be hypermethylated in a biological sample obtained from the patient.

In accordance with embodiments of the methods described here, the step of assaying a biological sample from the patient to determine the DNA methylation status of at least one CpG site includes detecting one or more 5-methylcytosine nucleotides in genomic DNA obtained from the sample. In embodiments, the step of assaying may further include one or more of isolating genomic DNA from the biological sample, treating the genomic DNA with bisulfite, and subjecting the genomic DNA to a polymerase chain reaction (DNA).

In embodiments, the sample is collected from the patient prior to undergoing a surgical procedure.

In embodiments, the perioperative pain is selected from preoperative pain, acute postoperative pain, and chronic postoperative pain. In embodiments, the perioperative pain is chronic postoperative pain.

In embodiments, the at least one CpG site at the PARK16 locus is identified in Table 1 below. In embodiments, the at least one CpG site at the PARK16 locus is selected from the RAB7L1 CpG sites designated cg16031515 and cg16031515 and the PM20D1 CpG sites designated cg14893161, cg12898220, cg11965913, cg07167872, cg16334093, and cg0715783 in Table 1 below.

TABLE 1
CG sites, methylation quantitative trait loci and chronic
postsurgical pain associations at the PARK16 locus.

| | | Location | | | p-value: | Difference | DNAm | |
|---|---|---|---|---|---|---|---|---|
| | | genome | Relation | | DNAm | in beta: | Step 2^{‡} | |
| | | build | to CpG | | step 1^{†} | CPSP | p-value | Snp- |
| CpG | CHR | 37/Hg19 | island | Gene | beta | yes vs no | DNAm-CPSP | CpG |
| cg16031515 | 1 | 205743344 | N_Shore | RAB7L1 | 0.004 | −0.106 | 0.005 | N |
| cg26418147 | 1 | 205743515 | N_Shore | RAB7L1 | 0.003 | −0.073 | 0.002 | N |
| cg14893161 | 1 | 205819251 | S_Shore | PM20D1 | 0.018 | 0.129 | 0.007 | N |
| cg12898220 | 1 | 205819356 | S_Shore | PM20D1 | 0.039 | 0.13 | 0.007 | N |
| cg11965913 | 1 | 205819406 | S_Shore | PM20D1 | 0.009 | 0.157 | 0.007 | N |
| cg07167872 | 1 | 205819463 | S_Shore | PM20D1 | 0.02 | 0.152 | 0.003 | N |
| cg16334093 | 1 | 205819600 | S_Shore | PM20D1 | 0.029 | 0.099 | 0.005 | N |
| cg07157834 | 1 | 205819609 | S_Shore | PM20D1 | 0.017 | 0.099 | 0.005 | N |

| | | | | | | | LL | UL | |
|---|---|---|---|---|---|---|---|---|---|
| | Risk | | | | | Odds | 95% | 95% | p- |
| meQTL | allele | CHR | Location | Function | Gene | ratio | CI | CI | value |
| rs16856110 | G | 1 | 205631767 | intronic | SLC45A3 | 0.218 | 0.055 | 0.870 | 0.031 |
| rs11240547 | A | 1 | 205632932 | intronic | SLC45A3 | 0.387 | 0.159 | 0.945 | 0.037 |
| rs2793374 | A | 1 | 205647508 | intronic | SLC45A3 | 3.626 | 1.456 | 9.030 | 0.006 |
| rs823105 | A | 1 | 205657570 | intergenic | SLC45A3; | 4.032 | 1.611 | 10.090 | 0.003 |
| | | | | | NUCKS1 | | | | |
| rs1172198 | A | 1 | 205662718 | intergenic | SLC45A3; | 4.679 | 1.755 | 12.470 | 0.002 |
| | | | | | NUCKS1 | | | | |
| rs823096 | A | 1 | 205679887 | intergenic | SLC45A3; | 4.051 | 1.592 | 10.310 | 0.003 |
| | | | | | NUCKS1 | | | | |
| rs823130 | A | 1 | 205714372 | intronic | NUCKS1 | 5.944 | 1.933 | 18.270 | 0.002 |
| rs4951261 | C | 1 | 205717823 | intronic | NUCKS1 | 0.338 | 0.136 | 0.842 | 0.020 |
| rs823114 | A | 1 | 205719532 | upstream | NUCKS1 | 2.881 | 1.252 | 6.630 | 0.013 |
| rs11240565 | A | 1 | 205722958 | intergenic | NUCKS1; | 0.376 | 0.157 | 0.902 | 0.028 |
| | | | | | RAB29 | | | | |
| rs708723 | A | 1 | 205739266 | UTR3 | RAB29 | 3.194 | 1.345 | 7.586 | 0.009 |
| rs1772159 | A | 1 | 205759195 | UTR3 | SLC41A1 | 6.020 | 1.907 | 19.000 | 0.002 |
| rs960603 | A | 1 | 205812614 | intronic | PM20D1 | 4.866 | 1.382 | 17.130 | 0.014 |

^{†}Step 1 (DNA methylation association): DNAm (DNA methylation) beta values = age + sex + race + CPSP + SVs (surrogate variables).
^{‡}Step 2 (DNAm-CPSP association): CPSP = DNAm (beta values).
meQTLs that have increase risk for CPSP (odds ratio >1) are not shaded in the lower table.
CHR: Chromosome.; CpG: Cytosine-guanine dinucleotide; CPSP: Chronic postsurgical pain; meQTL: Methylation quantitative trait loci.

In embodiments, the biological sample is a blood sample. For assaying DNA methylation, the blood sample is preferably a sample of whole blood, or one containing blood cells such as leukocytes and erythrocytes. In embodiments where the assay is for the expression of one or more cytokines in peripheral blood, the biological sample is preferably a serum sample.

In embodiments, the patient identified as susceptible to CPSP is administered a therapeutic agent selected from a demethylating agent and an inhibitor of the repressor element-1 silencing transcription factor (REST). In embodiments the agent is administered before or after a surgical procedure is performed on the patient. In embodiments, the demethylating agent is selected from procaine, zebularine and decitabine, or a combination of two or more of the foregoing. In embodiments, the demethylating agent is zebularine, decitabine, or a combination of two or more of the foregoing.

In embodiments, the biological sample is assayed by a method comprising isolation of genomic DNA from the biological sample, for example a sample of whole blood or serum. In embodiments, the biological sample is assayed by a method comprising, or further comprising, pyrosequencing. In embodiments, the pyrosequencing comprises two or more rounds of a polymerase chain reaction.

In embodiments, the patient is a pediatric patient.

### BRIEF DESCRIPTION OF THE DRAWINGS

The foregoing and other advantages and features of the disclosure will become apparent upon reading the following detailed description with reference to the accompanying figures and drawings.

FIG. 1 is a set of graphs illustrating an exemplary analysis workflow. Red text indicates tests; blue text indicates results.

FIG. 2 is a graph illustrating exemplary causal inference test steps. Omnibus test: intersection/union test for p-value of causal interference test.

FIGS. 3A-3B are a set of graphs illustrating exemplary enriched gene regulatory mechanisms within the 127 CpGs that causally mediate associations between SNPs and CPSP. FIG. 3A shows epigenetic markers enriched within these regions—histone marks, expression trait quantitative loci, and chromatin states. The X-axis indicates the significance (−log p-value), using the RELI software package (see Methods). The size of each circle indicates the fold-enrichment relative to background. Chromatin states are based on combinations of histone marks using the ChromHMM tool and data from RoadMap Epigenomics: 12 EnhBiv, bivalent enhancer; 2 TssAFlnk, region flanking an active transcription start site; 3 TxFlnk, transcribed regions; 7 Enh, enhancer. FIG. 3B shows transcription factor binding site motifs enriched within the DNA sequences of these regions. The Y-axis indicates regulatory proteins (e.g., transcription factors), in decreasing order of significance.

FIG. 4 is a graph illustrating exemplary differentially methylated CpG sites-meQTL pairs associated with chronic post-surgical pain in the PARK16 locus.

FIGS. 5A-5D are a set of graphs illustrating exemplary DNA methylation-meQTL associations. FIGS. 5A and 5B show association of rs708723 (RAB29) genotypes with differential DNA methylation at RAB7L1 (cg16031515) and PM20D1 (rs16334093) CpG sites respectively. FIGS. 5C and 5D show association of rs960603 (PM20D1) genotypes with differential DNA methylation at RAB7L1 (cg16031515) and PM20D1 (rs16334093) CpG sites respectively. In all panels, DNA methylation beta values for CpG sites are represented on y-axis and genotypes (AA, AG and GG) on the x-axis. In FIGS. 5A and 5C, red dots represent DNA methylation values in CPSP phenotypes while blue dots represent controls. CPSP phenotypes are more represented in AG and AA genotypes for both meQTLs compared to wild type GG. This is consistent with odds ratios for CPSP being 3.194 (95% CI 1.345-7.586; p=0.009) for rs708723 (risk allele A), and 4.866 (95% CI 1.382-17.310); p=0.014 for rs960603 (risk allele A). DNA methylation values are represented as box and whisker plots in FIGS. 5B and 5D, with colors denoting different CpG sites. Red plots in FIGS. 5B and 5D show that DNA methylation at cg16031515 (RAB7L1) was significantly lower for higher risk genotypes (AG and AA) compared to GG genotypes for both rs708723 and rs960603 (cg16031515 vs rs708723 genotypes: beta=−0.105, t-stat=−5.584, p<0.001 and cg16031515 vs rs960603: beta=−0.145, t-stat=5.923, p<0.001). Blue plots in FIGS. 5B and 5D show DNA methylation at cg16334093 (PM20D1) was significantly higher for risk genotypes (AG and AA) compared to GG for both variants (cg16334093 vs rs708723: beta=0.154; t-stat=6.820, p<0.001; cg16334093 vs rs960603 genotypes: beta=0.212, t-stat=7.232, p<0.001). The direction of associations with DNA methylation at RAB7L1 and PM20D1 with meQTLs associated with higher CPSP risk was consistent for all meQTLs on PARK16. This suggests that association of RAB29/RAB7L1 and PM20D1 meQTLs with CPSP risk is mediated by DNA methylation at CpG sites on these genes. *p<0.05; **p<0.001.

FIGS. 6A-6B are a set of graphs illustrating an exemplary mediation model showing mediation of meQTL association with CPSP by DNAm. Mediation model denoting mediation by DNAm at CpG sites on two genes (RAB7L1 and PM20D1) of association of meQTLs rs823114 G_A (NUCKS1) (FIG. 6A), rs960603 G_A (PM20D1) and rs708723 (RAB7L1) (FIG. 6B) with CPSP. Odds ratios for CPSP (all meQTLs are associated with increased Odds for CPSP) are presented. Causal inference test was significant for all these interactions. The direction of association of meQTL on DNAm and DNAm on CPSP are denoted by +(positive) and − (negative) signs. In FIG. 6B, the red font indicates specific additional CpG sites affected by meQTL rs708723. Net effect of all meQTLs was to decrease DNAm at RAB7L1 sites and thus decreased the protective effect of RAB7L1 CpG DNAm on CPSP, and to increase DNAm at PM20D1 associated with increased risk for CPSP. CPSP: Chronic post-surgery pain; DNAm: DNA methylation.

### DETAILED DESCRIPTION

The present disclosure is based, in part, on associations between epigenetic modifications in the genomic DNA of certain genes and preoperative pain, acute postoperative pain, and chronic postoperative pain following surgery. These findings allow for the identification of patients who are likely to be particularly susceptible to perioperative pain, especially acute and chronic postoperative pain. The ability to identify such patients allows for the development of targeted prevention and treatment regimens for acute and chronic postoperative pain.

In the context of the present disclosure, the term “CpG site” refers to a site in genomic DNA where a cytosine nucleotide is followed by a guanine nucleotide when the linear sequence of bases is read in its 5 prime (5′) to 3 prime (3′) direction. The ‘p’ in “CpG” refers to a phosphate moiety and indicates that the cytosine and guanine are separated by only one phosphate group. A status of “methylated” in reference to a CpG site refers to methylation of the cytosine of the CpG dinucleotide to form a 5-methylcytosine.

In the context of the present disclosure, the terms “acute postoperative pain” and “chronic postoperative pain” are synonymous, respectively, with the terms “acute postsurgical pain” and “chronic postsurgical pain”. The term “chronic postsurgical pain” may be abbreviated “CPSP”. In this context, the term “chronic” refers to pain that persists for more than two or three months after surgery. Likewise, the term “acute” refers to pain occurring within the first two months after surgery.

In the context of the present disclosure, the term ‘patient’ refers to a human subject and a patient who is “susceptible” is one who is predisposed to suffering from perioperative pain, especially acute and chronic postsurgical pain. The identification of such patients according to the methods described herein is intended to provide for more effective personalized pain management and, in embodiments, for the targeted prevention and/or treatment of acute and/or chronic postsurgical pain. The patient identified as susceptible to perioperative pain or as susceptible to having an atypical perioperative anxiety response may be administered an agent to mitigate that susceptibility, such as a demethylating agent or an inhibitor of the repressor element-1 silencing transcription factor (REST). In embodiments, the demethylating agent may be selected from procaine, zebularine and decitabine, or a combination of two or more of the foregoing. In embodiments, the demethylating agent is zebularine, decitabine, or a combination of two or more of the foregoing.

In accordance with embodiments of the methods described here, the biological sample from the patient which is used to isolate genomic DNA and determine methylation status is a blood sample. In these embodiments, blood is used as a proxy for the target tissue, brain, because brain tissue is generally inaccessible in the clinical context in which the present methods are performed. The use of blood as a substitute for various target tissues has been validated for example, by ChIP assay findings showing similar transcription factors at the identified CpG sites across tissues and regulatory regions in brain tissue relevant to pain, which may be indicative of methylation at these sites having an effect on expression. Last but not least, methylation profiles derived from 12 tissues were compared in a previous study and found to be highly correlated between somatic tissues (Fan H et al., Zhonghua Yi Xue Yi Chuan Xue Za Zhi 2015; 32(5):641-645). Davies et al. also reported that inter-individual variation in DNA methylation was reflected across brain and blood, indicating that peripheral tissues may have utility in studies of complex neurobiological phenotypes (Davies M N et al., Genome Biol. 2012; 13(6):R43). For example, a comparison of methylation profiles of human chromosome 6 derived from different twelve tissues showed that CpG island methylation profiles were highly correlated (Fan S et al., Biochem. Biophys. Res. Comm. 2009; 383(4):421-425). More recently, some inter-individual variation in DNA methylation was found to be conserved across brain and blood, indicating that peripheral tissues such as blood can have utility in studies of complex neurobiological phenotypes (Davies M N et al., Genome Biol. 2012; 13(6):R43).

In accordance with embodiments of the methods described here, the methylation status at a genomic site, for example, at a CpG site as described herein, is binary, i.e., it is either methylated or unmethylated. In some embodiments where multiple CpG sites are assayed, if at least one CpG site is methylated the region may be designated as methylated according to the claimed methods. This is because even if only one of several possible sites is methylated, if that site is a critical one for gene expression, its methylation may be sufficient. In other embodiments, where more than one of several possible CpG sites in a genomic region is methylated. the region may be designated as methylated or hypermethylated. Likewise, where more than one of several CpG sites is unmethylated, the region may be designated as hypomethylated. In embodiments, where the genomic region assayed contains three or more CpG sites, the designation of “hypermethylated” or “hypomethylated” may be based upon the methylation status of a simple majority of the CpG sites in the genomic region assayed.

### Methods of Assaying DNA Methylation Status

Embodiments of the methods described here include assaying a patient's genomic DNA to determine the DNA methylation status at one or more CpG sites in a plurality of genes described infra.

As noted above, a status of “methylated” in reference to a CpG site refers to methylation of the cytosine of the CpG dinucleotide to form a 5-methylcytosine. Accordingly, methods of determining the DNA methylation status at one or more CpG sites in a genomic region of DNA generally involve detecting the presence of a 5-methylcytosine at the site, or multiple 5-methylcytosine in the region of interest. The determination of DNA methylation status can be performed by methods known to the skilled person. Typically such methods involve a determination of whether one or more particular sites are methylated or unmethylated, or a determination of whether a particular region of the genome is methylated, unmethylated, or hypermethylated, through direct or indirect detection of 5-methylcytosine at a particular CpG site, or in the genomic region of interest.

Whole-genome methylation can be detected by methods including whole-genome bisulfite sequencing (WGBS), high-performance liquid chromatography (HPLC) coupled with tandem mass spectrometry (LC-MS/MS), enzyme-linked immunosorbent assay (ELISA)-based methods, as well as amplification fragment length polymorphism (AFLP), restriction fragment length polymorphism (FRLP) and luminometric methylation assay (LUMA).

Generally, in the context of the methods described herein, the methylation status of one or more specific CpG sites is determined. Suitable methods may include bead array, DNA amplification utilizing a polymerase chain reaction (PCR) followed by sequencing, pyrosequencing, methylation-specific PCR, PCR with high resolution melting, cold-PCR for the detection of unmethylated islands, and digestion-based assays. Bisulfite conversion is typically an initial step in these methods. Accordingly, in embodiments, the method for assaying DNA methylation status in accordance with the present disclosure may include a step of bisulfite conversion, for example, a step of treating a sample of genomic DNA with bisulfite thereby converting cytosine nucleotides to uracil nucleotides except where the cytosine is methylated.

In embodiments, the step of assaying DNA methylation status comprises pyrosequencing. The analysis of DNA methylation by pyrosequencing is known in the art and can be performed in accordance with published protocols, such as described in Delaney et al., (Delaney et al., In: Methods Mol. Biol. Ed. Albert C. Shaw, 2015; vol. 1343 (Chapter 19): pp. 249-264; Springer Humana Press, NY). This technique detects single-nucleotide polymorphisms which are artificially created at CpG sites through bisulfite modification of genomic DNA, which selectively converts cytosine to uracil except where the cytosine is methylated, in which case the 5-methylcytosine is protected from deamination and the CG sequence is preserved in downstream reactions. Generally, the method comprises treating extracted genomic DNA with bisulfite and amplifying the DNA segment of interest with suitable primers, i.e., using a PCR-based amplification.

In accordance with the methods described here, a human patient identified as at high risk or increased risk of CPSP may be treated with a DNA methylation modifying agent. Exemplary DNA methylation modifying agents are described below.

### Demethylating Agents

DNA demethylating agents inhibit DNA methyltransferases (DNMTs) such as DNMT1, which is responsible for the maintenance of methylation patterns after DNA replication, DNMT3A, and DNMT3B, each of which carries out de novo methylation.

In accordance with certain embodiments of the methods described here, a patient identified as susceptible to perioperative pain based on the patient's methylation status as described herein may be administered one or a combination of two or more demethylating agents, for example, as part of a personalized pain management regimen.

In embodiments, a demethylating agent administered in accordance with embodiments of the methods described here may be a nucleoside-like DNMT inhibitor or a non-nucleoside DNMT inhibitor.

In an embodiment, the agent is a nucleoside-like DNMT inhibitor. In embodiments, the nucleoside-like DNMT inhibitor is selected from azacytidine (VIDAZA™), and analogs thereof, including 5-aza-2′-deoxycytidine (decitabine, 5-AZA-CdR), 5-fluoro-2′-deoxycytidine, and 5,6-dihydro-5-azacytidine. In embodiments, the nucleoside-like DNMT inhibitor is selected from pyrimidine-2-one ribonucleoside (zebularine).

In an embodiment, the agent is a non-nucleoside-like DNMT inhibitor. In embodiments, the agent is an antisense oligonucleotide. In embodiments, the antisense oligonucleotide is MG98, a 20-base pair antisense oligonucleotide that binds to the 3′ untranslated region of DMNT1, preventing transcription of the DNMT1 gene. In embodiments, the non-nucleoside-like DNMT inhibitor is RG108, a small molecule DNA methylation inhibitor (Graca I et al., Curr. Pharmacol. Design. 2014 20:1803-11).

### REST Inhibitors

In accordance with embodiments of the methods described here, a patient identified as susceptible to perioperative pain based on the patient's methylation status as described herein, including a patient identified as susceptible to perioperative pain or hyperalgesia, may be administered an inhibitor of the repressor elements-1 silencing transcription factor (REST). In embodiments, the REST inhibitor is denzoimidazole-5-carboxamide derivative (X5050) (Charbord J et al., Stem Cells 2013; 20:1803-1811).

### Target Population

In embodiments of the methods described here, the methods are directed to a target population of patients in need of prophylaxis or treatment of perioperative pain. For example, in embodiments the subject in need is one who is scheduled to undergo a surgical procedure or one who has recently undergone such procedure. In embodiments, the target patient population may be further defined as discussed below. In the context of the methods described here, the term “patient” refers to a human subject. In embodiments, the term may more particularly refer to a human subject under the care of a medical professional.

In embodiments, the target patient population may be further defined by sex, age, or self-reported human population or ethnic group. In embodiments, the patient is an adolescent, as that term is understood by the skilled medical practitioner. In embodiments, the patient is female. In embodiments, the patient is Caucasian or white, as those terms are commonly understood in defining ancestry among humans.

### Kits

Kits useful in the methods disclosed here comprise components such as primers for nucleic acid amplification, hybridization probes, means for analyzing the methylation state of a deoxyribonucleic acid sequence, and the like. The kits can, for example, include necessary buffers, nucleic acid primers, and reagents for detection of methylation, as well as suitable controls, including for example bisulfite conversion controls, such as a bisulfite-treated DNA oligonucleotide of known sequence, and template free negative controls for pyrosequencing, as well as necessary enzymes (e.g. DNA polymerase), and suitable buffers.

In some embodiments, the kit comprises one or more nucleic acids, including for example PCR primers and bisulfite-treated DNA for use as a control, for use in the detection of the methylation status of one or more of the specific CpG sites identified herein, as well as suitable reagents, e.g., for bisulfite conversion, for amplification by PCR and/or for detection and/or sequencing of the amplified products.

In embodiments, the kit comprises a set of PCR primers for detecting the methylation status of one or more of the CpG sites identified herein. In embodiments, the kit comprises at least two sets of primers, long and nested.

In certain embodiments, the kit further comprises a set of instructions for using the reagents comprising the kit.

Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, NY 2001); March, Advanced Organic Chemistry Reactions, Mechanisms and Structure 5^{th} ed., J. Wiley & Sons (New York, NY 2001); and Sambrook and Russel, Molecular Cloning: A Laboratory Manual 3rd ed., Cold Spring Harbor Laboratory Press (Cold Spring Harbor, NY 2001) provide one skilled in the art with a general guide to many of the terms used in the present application.

All percentages and ratios used herein, unless otherwise indicated, are by weight. Other features and advantages of the present disclosure are apparent from the different examples set forth below. The examples illustrate different components and methodologies useful in practicing aspects of the present disclosure. The examples do not limit the claimed disclosure. Based on the present disclosure the skilled artisan can identify and employ other components and methodologies useful for practicing the methods described here.

### EXAMPLES

### Example 1. Methylation Quantitative Trait Locus Analysis of Chronic Postsurgical Pain Uncovers Epigenetic Mediators of Genetic Risk

### Summary

Overlap of pathways enriched by single nucleotide polymorphisms and DNA-methylation underlying chronic postsurgical pain (CPSP), prompted pilot study of CPSP-associated methylation quantitative trait loci (meQTL). Materials & methods: Children undergoing spine-fusion were recruited prospectively. Logistic-regression for genome- and epigenome-wide CPSP association and DNA-methylation-single nucleotide polymorphism association/mediation analyses to identify meQTLs were followed by functional genomics analyses. Results: CPSP (n=20/58) and non-CPSP groups differed in pain-measures. Of 2753 meQTLs, DNA-methylation at 127 cytosine-guanine dinucleotides mediated association of 470 meQTLs with CPSP (p<0.05). At PARK16 locus, CPSP riskmeQTLs were associated with decreased DNA-methylation at RAB7L1 and increased DNA-methylation at PM20D1. Corresponding RAB7L1/PM20D1 blood eQTLs (GTEx) and cytosine-guanine dinucleotide-loci enrichment for histone marks, transcription factor binding sites and ATAC-seq peaks suggest altered transcription factor-binding. Conclusion: CPSP-associated meQTLs indicate epigenetic mechanisms mediate genetic risk.

Chronic postsurgical pain (CPSP) is a significant problem in children, with an incidence ranging from 14.5 to 38% [1-3]. Both genetic and environmental factors influence the risk for developing CPSP [4,5]. See a recent systematic review of genetic variants associated with CPSP [6]. It was found that well powered genome-wide association studies (GWAS) are scarce in CPSP, [7,8] and many of the SNPs described in genetic association studies [9-12] are located in noncoding regions, rendering functional interpretation difficult. The impact of environmental factors on the development of CPSP underscores the role of epigenetic mechanisms in the pathogenesis of acute to chronic pain transitions [13-15]. One widely studied epigenetic mechanism is DNA methylation. DNA methylation occurs primarily at cytosine-guanine dinucleotide (CpG) sites. DNA methylation influences gene expression without alterations to the underlying DNA sequence. Previous epigenome-wide study in children undergoing spine fusion surgery showed that DNA methylation at CpG sites in genes enriching opioid, dopaminergic and GABA pathways was associated with CPSP [16, 17]. A systems biology guided study delineated genetic pathways involved in the pathophysiology of CPSP [18]. Thus, it was hypothesized that by integrating DNA methylation analyses with genetic studies, the mediating molecular mechanisms underlying CPSP can be elucidated [19].

In the postgenome-wide association studies era, there is considerable interest in research focused on the influence of genetic polymorphisms (SNPs) on the level of DNA methylation at proximal CpG sites, also referred to as methylation quantitative trait loci (meQTLs) [20,21]. Identification of meQTLs provides putative mechanistic evidence for the role of noncoding genetic risk alleles with no known function. Additionally, they help identify molecular mechanisms—DNA methylation at specific CpG sites—that mediate the association between genotype and phenotype [22]. This is supported by the findings that meQTLs are located often at regulatory elements than expected by chance, providing credence to their ability to impact phenotype/disease risk by influencing transcription factor (TF) binding, chromatin conformation and gene expression [23, 24]. This approach has been successfully utilized in a number of conditions, including schizophrenia, alcohol dependence, obesity, cancer, rheumatoid arthritis and metabolic traits [25-29].

This Example illustrates an exemplary pilot study aimed to identify meQTLs in blood that affect predisposition to CPSP through DNA methylation in a surgical cohort of adolescents with idiopathic scoliosis undergoing spine surgery. Understanding epigenetic-mediating mechanisms is crucial because while genetic variation (meQTL) cannot currently be modified, DNA methylation may be a modifiable risk factor.

### Methods

An observational prospective cohort study was conducted in adolescents with idiopathic scoliosis undergoing posterior spine fusion using standard surgical techniques, anesthetic and pain protocols. The studies are approved by the institutional review board. Written informed consent from parents and assent from children was obtained prior to enrollment. Pilot epigenomewide and candidate DNA methylation study results from this cohort have been previously published [16, 17].

### Inclusion Criteria

Children aged 10-18 years of American Society of Anesthesiologists physical status less than or equal to two (mild systemic disease) with a diagnosis of idiopathic scoliosis and/or kyphosis, scheduled to undergo elective spinal fusion.

### Exclusion Criteria

Females who were pregnant or breastfeeding; subjects with a diagnosis of chronic pain; opioid use in the past 6 months; hepatic/renal disease and/or developmental delays.

### Data Collection

Preoperatively, data were obtained on demographics (sex, age, race), weight, pain scores (Numerical Rating Scale/0-10; NRS) [30] and home medications. Questionnaires to assess anxiety sensitivity (childhood anxiety sensitivity index) [31] were administered preoperatively. All patients received total intravenous anesthesia (propofol and remifentanil) and midazolam in the intraoperative period followed postoperatively by standardized doses of patient-controlled analgesia (morphine or hydromorphone). Postoperatively, pain scores (every four hours) and doses of morphine equivalents administered on postoperative days one and two were recorded. At 6-12 months posthospital discharge, patients were asked to rate their average pain score (NRS) over the previous week. Data about psychologic questionnaires collected, anesthetic medications and pain (nature and location) for a larger cohort have been presented previously [32].

The primary outcome was incidence of CPSP, determined to be positive if a pain score >3/10 was reported on an 11-point NRS (range 0-10) 6-12 months after surgery. This cut-off was chosen because NRS pain scores >3 (moderate/severe pain) at three months have been described as a predictor for persistence of pain, associated with functional disability [33]. The NRS for pain intensity has been validated as a pain measure in children aged 7-17 years [30]. Occurrence of infection and malignancy in the interim period leading/contributing to chronic pain was ruled out.

### Genotyping & Measurement of DNA Methylation

Blood samples were collected in EDTA tubes before surgery. DNA was isolated on the same day, and frozen at −80° C. Genotyping was done using the Illumina Human Omni5 v41-0 array (14 patients), Human Omni5Exome v41-1 (21 patients) and InfinumOmni5-4-v1 (39 patients). Arrays were changed due to availability of new array with more SNPs. SNPs from autosomal chromosomes were selected for analysis and annotated using ANNOVAR software [34].

All samples passed 95% threshold for call rates at genotype and individual levels. Genetic data was assessed for Hardy-Weinberg equilibrium by means of a goodness of fit χ² test with threshold for p-values 0.0001. Low frequency variants were also excluded—the threshold for minor allele frequencies was 10%. DNA methylation was measured using using Zymo EZ DNA Methylation Gold kit (Zymo Research, CA, USA), as described previously [16]. Data were then quality-controlled and preprocessed as previously described. Both beta and M values were obtained and used. To control for unwanted variation and potential batch effects, surrogate variables were obtained using the R package ‘sva’ and included as co-variables in relevant analyses. Illumina annotation was used for data interpretation, for example, probe location within genes, CpG islands and shores, and regulatory features (see the World Wide Web site at genome.ucsc.edu; UCSC Genome Bioinformatics, CA, USA).

### Data Analysis

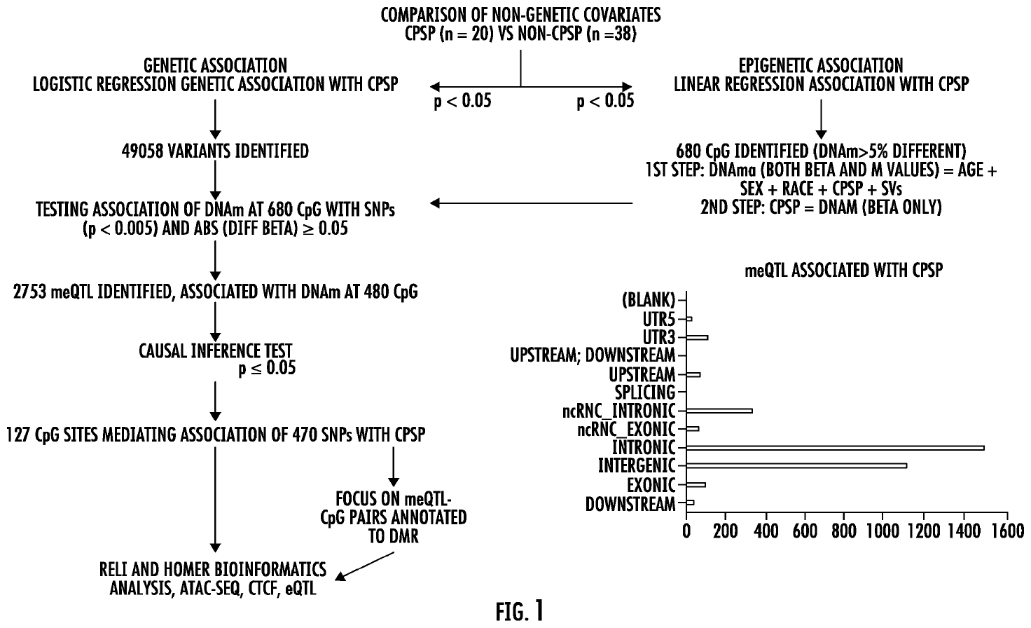

The clinical characteristics, demographics and pain variables of the cohort were described using mean (with standard deviation), median interquartile range and frequency (percentage) depending on data distribution. The CPSP and non-CPSP groups were compared for non-genetic factors including demographics, surgical duration and childhood anxiety sensitivity index using statistical methods appropriate for the distribution of the data. Factors significantly different between groups (p<0.05) were included as covariates for further genetic/epigenetic association analyses. Since preoperative pain and acute postoperative pain are correlated with CPSP as primary pain outcomes, with possible overlap of DNA methylation associations, they were not considered as co-variables in the multivariate genetic/DNA methylation models for CPSP. An overview of the analytic workflow is provided in FIG. 1.

### Genetic & DNA Methylation Association Analyses

Analyses were conducted using PLINK version 1.9 [35]. To identify genetic variants that were significantly associated with presence/absence of CPSP for each of the variants, logistic regression was performed, assuming additive genetic effects in which genotypes were numerically coded according to the number of minor alleles. Variants associated with CPSP at p<0.05 were selected for further analyses. DNA methylation association with CPSP: The beta and M values were first regressed against CPSP with age, sex, race and corresponding SVs controlled using the R package ‘limma’. CpG sites whose methylation significantly (p<0.05) differed by 5% between CPSP and non-CPSP were the steps selected. Associations between these CpG sites and CPSP were then confirmed by logistic regression using CPSP as the dependent variable. Only CpGs that showed significantly association in the logistic regression were used in subsequent analyses.

meQTL Analysis

Using the R package ‘MatrixEQTL’ [36], meQTLs were identified by regressing DNA methylation of the significant CpG sites against the genotypes of the genetic variants. For methylation levels of each SNP-CpG pair, linear regression was performed assuming additive genetic effects. Age, sex, race (White vs non-White) and SVs were adjusted as co-variables. meQTL were selected if DNA methylation (measured by both beta and M values) and genotype associated at p<0.05 level, and one copy of the minor allele led to a 5% change or more in DNA methylation.

### Causal Inference Test for Mediation Analysis

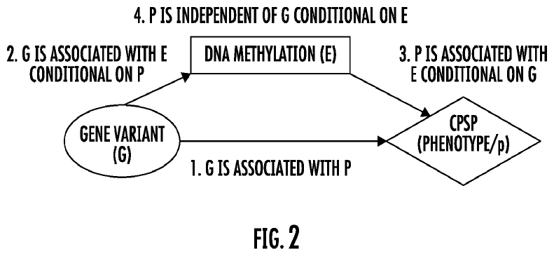

The causal inference test (CIT) is based on hypothesis testing rather than estimation, allowing the testable assumptions to be evaluated in the determination of statistical significance [37]. All meQTLs selected in the steps above were included in the CIT performed using the R package ‘CIT’ to select meQTLs whose association with CPSP was significantly mediated by DNA methylation (p<0.05). The steps are illustrated in FIG. 2 (see the World Wide Web site at cran.r-project.org/web/packages/cit/index.html) [38].

### Functional Genomics Analysis

To identify epigenetic marks and TF binding events that are enriched at 5mC sites, a previously described computational method was used [39], where the set of genomic locations with 5mC marks were overlapped with a large collection of functional genomics datasets from ENCODE [40], Roadmap Epigenomics [41] and chromatin immunoprecipitation (ChIP)-seq for histone marks [42].

All datasets were indexed by their genomic coordinates, which were used to intersect with the genomic coordinates of methylation sites of interest. Using RELI [43], the significance of such intersections was estimated by comparing to a null model, which consists of regions with 5mC (beta >0.2) but no significant association with CPSP (control) and those associated with CPSP (p >0.05 AND absolute paired difference <0.2). The expected intersection values from the null model was normally distributed. The model parameters were estimated and the significance of the observed number of intersections, for example, a Z-score and the corresponding p-value was calculated for the observed intersections of methylation sites of interest. This procedure controls for the count and sizes of the input loci and each individual dataset in the library. Genomic loci with 5mC marks were also examined using standard TF motif enrichment analysis, using HOMER motif enrichment algorithm [44] and human position weight matrix binding site models from the CisBP database [45] as described before [39].

Focus on meQTL-CpG pairs (significant by CIT) annotated to differentially methylated regions (DMR) by Illumina: due to the focus on meQTL, a DMR analysis was not conducted. Instead the focus was given to DMR annotations by Illumina which were enriched for meQTL-CpG pairs. HOMER/RELI findings overlapping CpG sites at these locations were evaluated. Genotype-Tissue Expression portal was used to identify correlations between genotypes of meQTLs and blood gene expression levels to determine if direction of effects on DNA methylation were consistent with reported computed expression quantitative trait loci (eQTLs). Assay for Transposase-Accessible Chromatin using sequencing (ATAC-seq) was conducted on isolated monocytes from one patient (Phenotype: CPSP) to show evidence for open accessibility in meQTL-CpG enriched DMR (by annotation) associated with CPSP. Using fresh blood (7 ml) collected preoperatively, white blood cells were purified by HESPAN sedimentation, aliquoted and viably frozen in serum/DMSO solution. This protocol produces >80% viable cells upon thawing. Since DNA accessibility is affected by cell composition, ATAC-seq was performed on CD14+ monocytes negatively selected using magnetic kits from Stem Cell. This cell subset preference is based on evidence of differential immune signatures correlated to pain in patients undergoing hip arthroplasty [46, 47]. Tn5 reaction was performed using OMNI-ATAC [48] protocol on 50 K magnetically purified cells. Libraries were amplified and submitted for Illumina sequencing at Novagene (PE150, ˜20M reads). Data analysis was performed using SciDAP platform (see the World Wide Web site at scidap.com, Datirium, LLC) [49]. Containerized CWL pipelines are available at the World Wide Web site of github.com/datirium/workflows. Briefly, trimmed ATAC reads were aligned to the human genome using Bowtie [50] extended to 9 bp, normalized to total mapped read number and displayed as coverage on IGV-JS genome browser built into SciDAP. MACS2 was used to identify islands of enrichment [51].

### Results

74 participants was recruited for the study. The mean age of participants was 14.4 years (standard deviation 1.7); they were mostly White (79.71) and female (85.1%). A description of pain and other variables is provided in Table 2. Follow-up for CPSP outcomes was successful for 58 of these subjects (loss to follow-up 16/74-21.6%). Incidence of CPSP in this cohort was 20/58 (34.50). Preoperative pain, acute postoperative pain and pain at 6-12 months were significantly higher in the CPSP group compared with the non-CPSP group. None of the other factors were identified to be significantly different by univariate analysis (p<0.05).

TABLE 2
Demographic, perioperative and pain data for the entire cohort, subjects
who developed chronic postsurgical pain (chronic postsurgical pain
yes) and those who did not (chronic postsurgical pain no).

| Variable | All (n = 74) | | CPSP no (n = 38) | | CPSP yes (n = 20) | | p-value |
|---|---|---|---|---|---|---|---|
| Age (year) | 14.4 ± 1.7 | | 14.4 ± 1.8 | | 14.9 ± 1.4 | | 0.24^{†} |
| Sex (male) | 11 | (14.9%) | 7 | (18.4%) | 1 | (5%) | 0.24^{‡} |
| Race (White) | 59 | (79.7%) | 31 | (81.6%) | 16 | (80.0%) | 1.00^{‡} |
| Weight (kg) | 53.7 | (50.4-57.2) | 53.7 | (50.8-58.5) | 53.8 | (50.0-58.0) | 0.88^{§} |
| CASI | 28.9 ± 5.5 | | 27.5 ± 5.1 | | 30.0 ± 5.5 | | 0.15^{†} |
| Preoperative pain | 0.0 | (0.0-0.0) | 0.0 | (0.0-0.0) | 0.0 | (0.0-2.0) | 0.010^{§} |
| score | | | | | | | |
| Surgical duration | 4.3 ± 1.1 | | 4.3 ± 1.0 | | 3.9 ± 1.2 | | 0.20^{†} |
| Pain AUC POD 1 | 207.0 ± 82.8 | | 185.6 ± 75.2 | | 242.0 ± 88.9 | | 0.024^{†} |
| and 2 | | | | | | | |
| Morphine dose | 1.3 | (0.9-1.9) | 1.3 | (1.0-1.8) | 1.5 | (1.1-2.3) | 0.22^{§} |
| POD 1 and 2 | | | | | | | |
| (mg/kg) | | | | | | | |
| Pain scores at 6-12 | 1.0 | (0.0-4.0) | 0.0 | (0.0-6.0) | 5.0 | (4.0-6.0) | <0.001^{§} |

months

^{†}t-test;
^{‡}Fisher's exact test;
^{§}Wilcoxon rank sum test.
AUC: Area under curve; CASI: Childhood Anxiety Sensitivity Index; POD: Postoperative day.

Genetic & DNA Methylation Association with CPSP

The following quality control—exclusion workflow was utilized: 121,301 SNPs from the sex chromosome, chromosome zero, mitochondrial, indels and other were excluded from analysis. SNPs that failed Hardy-Weinberg Equilibrium (p<0.0001) or had minor allele frequencies below 10% were excluded. There were 4,186,587 variants on the exome chip initially and 1,270,531 variants remained after exclusion by QC. A total of 49,058 SNPs were nominally associated with CPSP (p<0.05). The results are enumerated according to the analytic workflow in FIG. 1. Following QC, 842,148 CpG sites were deemed evaluable. DNA methylation (both beta and M values) at 680 CpG sites were found to be associated with CPSP (p<0.05 and >5% difference in beta values).

meQTL (SNP-CpG-DNA Methylation Association) Underlying CPSP

Pairwise association of the 49,058 variants with DNA methylation at 680 CpG sites identified 2753 meQTL with significant impact on the DNA methylation levels (≥5% change) at 480 CpG sites. Less than 100 of the meQTLs identified were exonic (see inset of FIG. 1), with the majority being located in intronic or intergenic regions.

### CIT Results

After CIT, 529 variant-CpG associations were found, with 127 unique CpG sites potentially mediating the genetic association of 470 variants with CPSP. The relation of the CpG sites location in relation to CpG islands per the UCSC browser was available for 57.5% of the 127 sites (26.0% were located in a CpG island, 12.6% in the north shore of the islands, 15.7% in the south shore and 1.6% each in north and south shelves. Details of CpG-meQTL pairs significant by CIT were collected. Descriptions of meQTL with SNP annotations from ANNOVAR, CpG site details, CIT results, association of DNA methylation at the site and Odds ratio for meQTL with CPSP are tabulated.

### Functional Genomics

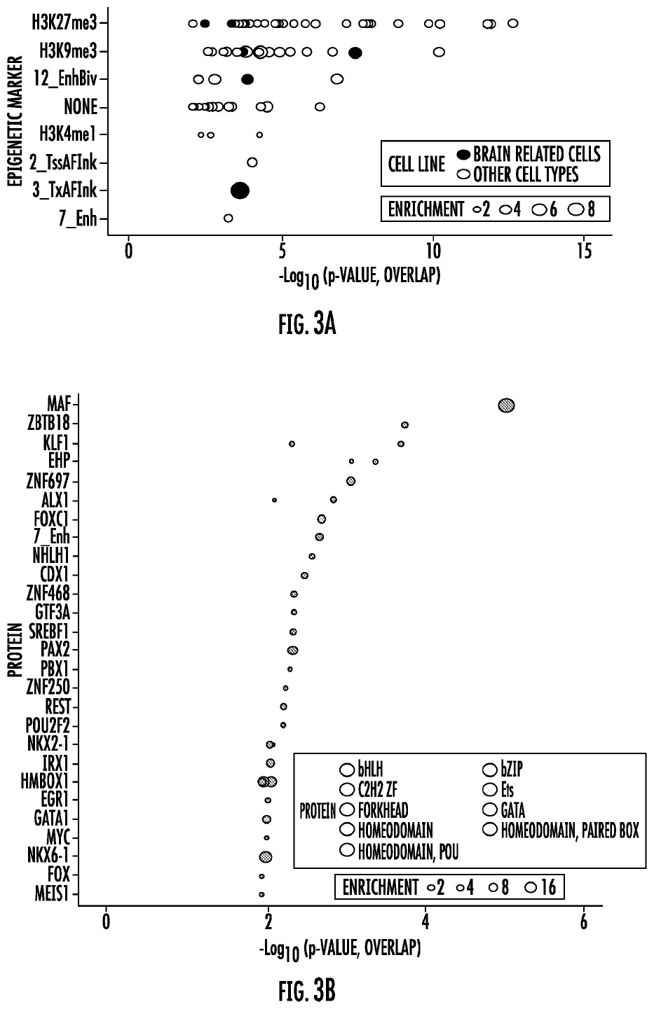

To better understand the potential functions of the 127 CpGs identified by the CIT, functional genomics analyses was used to identify enriched epigenetic markers (e.g., histone marks) and TF-binding motifs (see Methods). These analyses revealed strong enrichment for repressive (H3K27me3 and H3K9me3) and active (H3K4me1) histone marks, in addition to eQTLs, consistent with these regions being located within regulatory regions in brain-derived, and other tissues (FIG. 3A). Further analysis revealed enrichment for binding sites for particular TFs, including MAF, KLF1, FOXC1 and GATA1, at these loci (FIG. 3B).

### PARK16 Locus CpG-DNA Methylation Associations

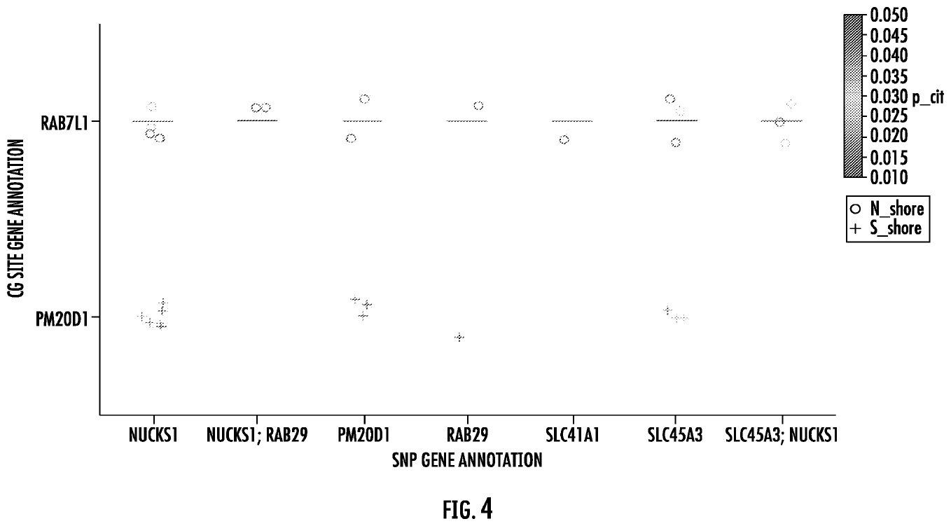

It's focused on meQTL-CpG DNA methylation pairs significant by CIT, annotated to DMR by Illumina. Interestingly, 18 of such 26 meQTL-CpG DNA methylation associations were between meQTLs at PARK16 locus and CpG sites on PARK16 locus (DNA methylation at RAB7L1 CpG sites annotated to a DMR, and CpG sites on PM20D1) (FIG. 4). PARK16 locus is a genetic region spanning five genes on chromosome 1q32. The five genes that make up the PARK16 locus include SLC45A3, NUCKS1, RAB7, RAB7L1, SLC41A1, and PM20D1.

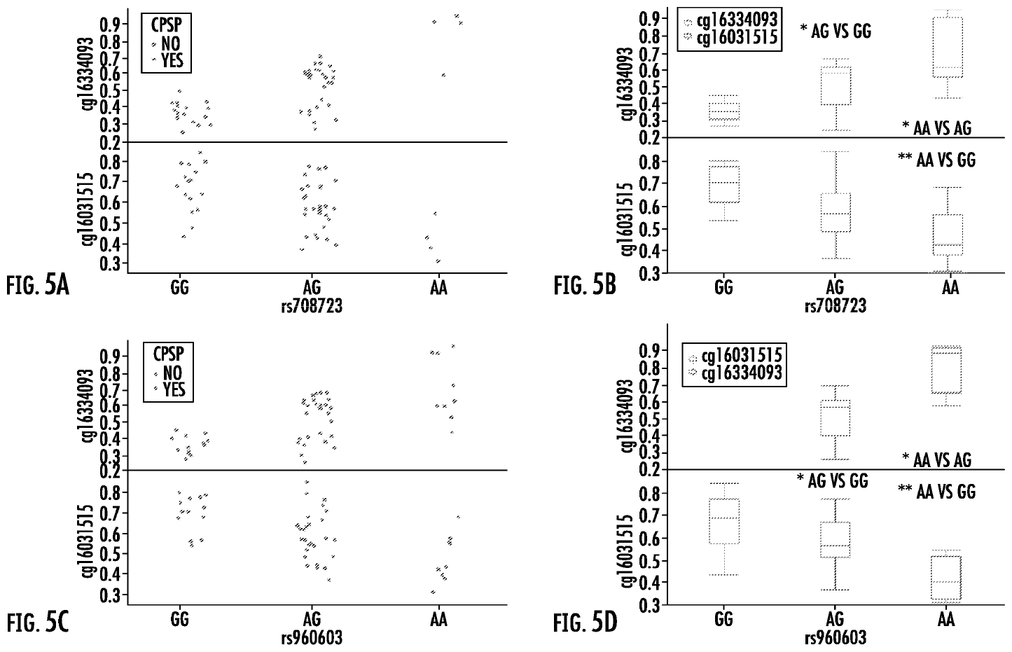

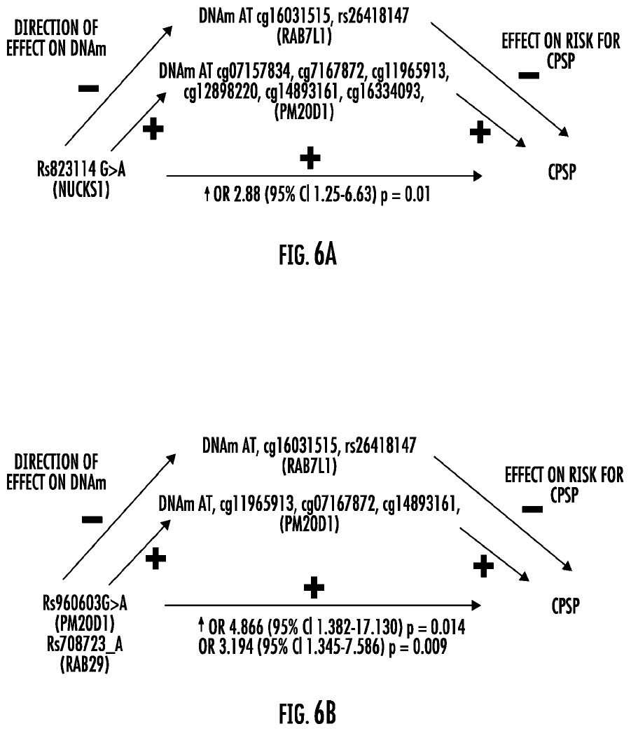

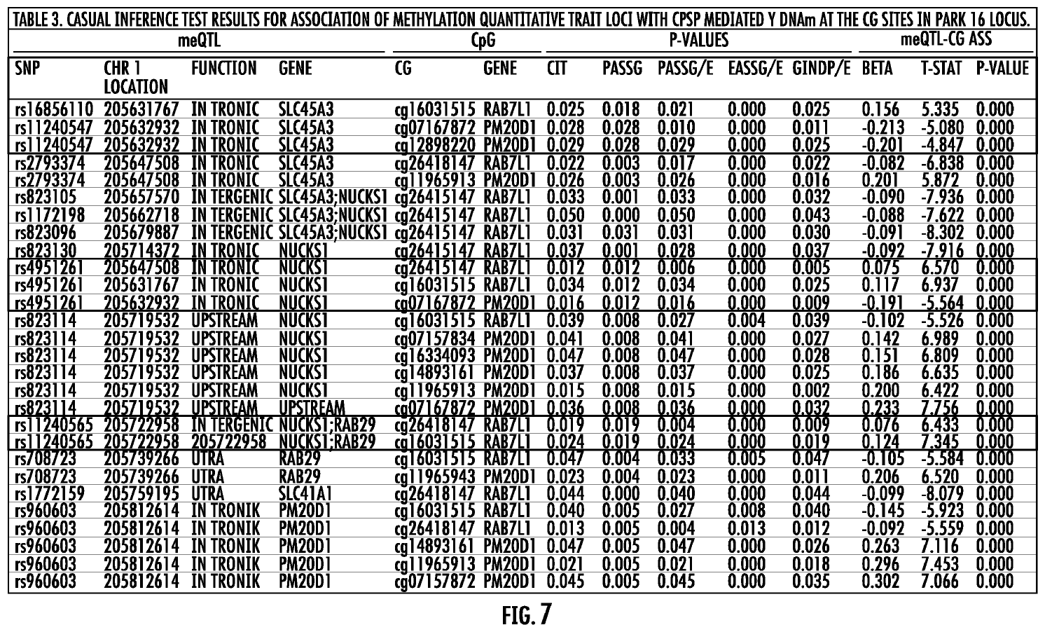

meQTLs on genes NUCKS1 (rs4951261, rs823114, rs823130, rs11240565), PM20D1 (rs960603), RAB29 (rs708723), SLC45A3 (rs16856110, rs2793374, rs11240547), SLC41A1 (rs1772159), SLC45A3/NUCKS1 (rs1172198, rs823096, rs823105) and ELK4/SLC45A3 (rs11589772) were associated with DNA methylation on CpG sites in the north shore of CpG islands on RAB7L1 and sites on south shore of CpG islands on PM20DL. The level of DNA methylation in shores has previously been shown to be more highly correlated with gene expression than that of CpG islands likely because of transcription machinery binding to nearby promoter CpG islands [52]. DNA methylation at CpG sites on RAB7L1 were uniformly hypomethylated and PM20D1 CpG sites were uniformly hypermethylated in association with high risk CPSP genotypes. Beta values for association of CpG-DNA methylation with CPSP and meQTL associated odds for CPSP risk in the PARK16 region are provided in Table 1. Representative meQTL (rs708723 (RAB29) and rs960603 (PM20D1) genotypes)-CpG (cg 16031515 on RAB7L1 and cg16334093 on PM20D1) DNA methylation with superimposed CPSP phenotypes are shown in FIGS. 5A-5D. Description of meQTL, the CpG sites whose DNA methylation they affect, p-values of CIT components, and association of meQTL with DNA methylation at the CG sites for the PARK16 locus are tabulated in Table 3. Examples of meQTL associations with CPSP mediated by CpG sites in PARK16 are illustrated in FIGS. 6A-6B. Directions of association of meQTL-DNA methylation at CpG sites and CPSP is consistent with risk genotypes. Higher PM20D1 DNA methylation mediates meQTL association with higher CPSP risk. Risk genotypes are associated with decreased DNA methylation at RAB7L1 CpG sites; however, since DNA methylation at RAB7L1 sites are independently protective of CPSP, hypomethylation at RAB7L1 mediates association of meQTL with higher risk for CPSP.

### Functional Genomics: PARK16

RELI analyses showed significant overlap (p<0.001) overlap for transcription factor binding sites (TFBS) (for GATA2, IKZF1 and INO80) at CpG sites with differential DNA methylation at PARK16 loci (RAB7L1 and PM20D1). These results indicate that these particular TFs might have altered binding events due to differential methylation. eQTLs for rs708723 (RAB7L1), rs960603 (PM20D1) and rs823114 (NUCKS1) genotypes accessed through G-TEx portal were collected and analyzed. Consistent with expected expression based on DNA methylation associations with meQTLs, significantly higher RAB7L1 and lower PM20D1 eQTLs associated with high risk genotypes, but no eQTLs for other genes in PARK16 (SLC41A1 and NUCKS1), were found.

### ATAC-Seq: PARK16

Chromatin accessibility peaks were identified in PARK16 locus genes overlapping mediating CpG sites as well as meQTLs associated with CPSP. For example, Chr1: 205819055-205819458 in PM20D1 shows a broad ATAC-seq peak overlapping 6 CpG sites (Chr1:205819251-205819609) indicating open chromatin regions amenable to be affected by DNA methylation changes leading to altered TF binding. Also shown are overlapping CTCF peaks in monocytes (CD14+ cells) from publicly available ENCODE dataset (EH002169, GSM1022659). This includes a previously reported CTCF peak at Chr1: 205,760,411-205,761,320 in the SLC41A1 gene transcription end site, about 17 kb from the CpG sites annotated to CpG sites on RAB7L1 (Chr1: 205743344-205743515) and about 60 kb away from the differentially methylated CpG sites enriching south shore of CpG islands on PM20D1 (Chr1: 205819251-205819609).

### Discussion

In this pilot study, by integrating genotype and DNA methylation analyses, 2753 putative meQTLs were identified associated with CPSP. In addition, DNA methylation at 127 CpG sites were found to mediate associations between 470 SNPs and CPSP. The PARK16 locus on Chromosome 1 was identified as a primary site with several meQTLCpG associations. The association of meQTLs annotated to genes in the PARK16 genetic locus with CPSP were found to be mediated by reprogramming-specific differentially methylated regions on two genes in the same region (RAB7L1 and PM20D1). meQTLs associated with risk of CPSP were consistently associated with decreased DNA methylation at RAB7L1 and increased DNA methylation at PM20D1 CpG sites. In the absence of cell- and tissue-specific experimental evidence, the functional data interpretation provides putative evidence that differential methylation at the CpG sites modulates binding of TFs and might represent the possible mechanism underlying the regulatory effects of some noncoding gene variants on CPSP development.

The genomic methylation pattern at PARK16 locus has been found to be associated with neurodegenerative conditions such as Parkinson's disease and Alzheimer's disease [53-57]. This is the first time this region is shown to be associated with molecular mechanisms linked to acute to chronic pain transition. Although there is no direct evidence for the role of PARK16 in chronic pain, it is known that parkinson's disease (PD) is associated with chronic pain and abnormal pain processing [58]. Dopamine deficit lowers multimodal pain thresholds and dopaminergic mechanisms underly pain, depression, and addiction [59]. Previous findings—differential DNA methylation in regulatory genomic regions enriching GABA and dopamine DARPP32 signaling pathways associated with CPSP and anxiety sensitivity in adolescents undergoing spine surgery—also suggested dopaminerelated emotion/reward contributes to behavioral maintenance of pain after surgery [16]. Notably, methylation changes have been described to be consistent in brain and blood for this genomic region, supporting the notion of using blood tissue DNA methylation as a proxy for DNA methylation changes in the brain [60].

DNA methylation is tissue specific and for brain-related phenotypes such as CPSP, careful consideration of tissue source for epigenetic analyses is important. Use of blood for DNA methylation studies in neural phenotypes is supported by blood-brain DNA methylation concordance studies which have observed high correlation of DNA methylation levels across tissues especially related to genetic influences [61, 62]. Also, a significant overlap of cis meQTLs (45-73%) and targeted CpG sites (31-68%) has been reported across brain prefrontal cortex, whole blood and saliva [29]. In fact, cross-tissue meQTLs are also enriched in cross-tissue eQTLs in association with schizophrenia [29]. meQTL maps and cross-tissue meQTLs have previously been examined using data of meQTL and corresponding CpG sites derived from infant cord, blood tissue from children, and publicly available brain tissue databases. It was found that in subjects with autism spectrum disorder, both peripheral blood and fetal brain were enriched for meQTLs [63]. Similar findings for overlap of, meQTL signals across adult brain and blood tissues [20], further suggest blood-derived meQTLs may serve as biomarkers and represent brain tissue SNP-DNA methylation relationships. This gives confidence in blood-based findings in relation to a neural phenotype [64]. The findings provide a basis for future investigation of brain and blood meQTLs for specifically pain phenotypes.

The PARK16 locus bears most of the major findings. There is prior evidence for the function of variants identified in PARK16 locus (rs960603 and rs708723) as meQTLs. The results show that rs960603 A allele on PM20D1 is associated with increased risk for CPSP and increased DNA methylation at CpG sites on PM20D1. The direction of meQTL-DNA methylation associations are aligned with previous studies which found an association between PM20D1 hypermethylation and Alzheimer's disease. PM20D1 methylation in human frontal brain cortex samples was shown to be dependent on the rs708727-rs960603 haplotype [53]. An allele-dependent correlation with PM20D1 promoter methylation (TT associated with increased DNA methylation) and further, that PM20D1 expression was inversely correlated with the methylation of its promoter, were observed. In fact, DNA methylation at the same CpG site (cg14893161; Chr 1: 205,819,251) is also affected by rs708723 and rs960603 in the instant study. Sanchez-Mut et al. also used ChIP assays and 3-C assays to show that meQTLs regulate long-range chromatin interaction of the RAB29-PM20D1 loci and reduce CTCF binding to 3C anchors in human frontal cortex samples with highly methylated at the PM20D1 promoter (cg14893161). In addition, it was demonstrated that transcriptionally silent chromatin state could be restored upon use of DNA demethylating agent 5-aza-2′-deoxycytidine, in turn affecting the chromatin loop and PM20D1 expression. Thus, in Alzheimer's disease, when PM20D1 CpG sites are non-methylated, an enhancer region 60 kb downstream of PM20D1 physically interacts with PM20D1 promoter via CTCF binding and favors PM20D1 transcription, which is protective; and hypermethylation is associated with pathology. Similarly, in CPSP, it is hypothesized that PM20D1 meQTL effects through hypermethylation at CpG sites increase risk for CPSP, although the mechanism is yet unknown.

A two stage meta-analysis found rs708723/1q32 T allele in RAB29 increased PD risk [57]. They tested whether association of rs708723 with gene expression and DNA methylation status of proximal transcripts or CpG sites respectively. They found correlations of T>C with increased expression of NUCKS1 (p=1.8×10⁻⁷) and decreased expression of RAB7L1 (p=7.2×10⁻⁴) in frontal cortex and cerebellar tissue, thus revealing potential biological consequences of this variant. The current study provides further evidence that association of phenotype risk (in this case, CPSP) with rs708723 A (or T) allele in RAB29 (Chr 1: 205739266) is mediated by decreased DNA methylation at CpG sites at RAB7L1 and increased DNA methylation at CpG sites at PM20D1 thus providing a molecular mechanism for the associations between SNP-gene expression. Another study investigating DNA methylation and gene expression in in frontal cortex and cerebellar regions in subjects with PD supports the current findings that higher odds for CPSP for meQTL rs823114 G >A on NUCKS1 was mediated through increased DNA methylation at CpG sites in PM20D1. This study reported that the PD risk allele (T) at rs823118 (in LD with rs823114) was associated with increased methylation at PM20D1. In addition, they showed PD associated decreased expression of NUCKS1 and increased transcription of RAB7L1 in brain tissues [65]. Thus, there seems to be consistency in mechanisms and roles of meQTLs/DNA methylation at PARK16 locus for CPSP and other neurologic conditions.

In addition, there is prior evidence for the role of the genes in the PARK16 locus in pain pathology. RAB7L1 is a small cytosolic GTPase belonging to the RAB-related GTP-binding protein subfamily [66] has been implicated in intracellular cell signaling processes and vesicle trafficking [67]. While in gene knockdown mice, significantly decreased neurite process length was found [56] overexpression was protective against alpha-syn-induced dopaminergic neuronal loss in animal models of PD [68]. This would suggest increased expression was protective for CPSP. However, gain-of-function mutations in RAB7 were proposed to cause Charcot-Marie-Tooth type 2B neuropathy, a disorder characterized by sensory loss [69] with increased lysosomal activity, autophagy and premature degradation of long axons due to trafficking of neurotrophic factors and prolonged mitogen-activated protein kinase activation in the long axons of peripheral sensory neurons [70]. Given the conflicting mechanisms for RAB7L1 in PD and Charcot-Marie-Tooth, there is much to be known about its mechanistic role in CPSP. Interestingly, PM20D1 codes for PM20D1, a factor secreted by thermogenic adipose cells that catalyzes both the hydrolysis and condensation of N-acyl amino acids, which have been found to play a role in nociception [71]. PM20D1-knockout mice were shown to have bidirectional changes in N-acyl amino acid levels in blood/tissues and influence late nociceptive behaviors in mice in formalin assay models [72]. This is aligned with the instant observations that increased DNA methylation of PM20D1 (decreased expression) increased CPSP risk. The differences in early and late nociceptive effects of PM20D1 may support the use of PM20D1 inhibitors for acute but not chronic pain management [72].

The key link between DNA methylation and gene expression is related to chromatic accessibility and TFBS. TFs play an important role in altering gene expression in response to injury, stress and other events influencing neuronal plasticity and pain [73]. Using bioinformatics enrichment methods to integrate chromatin state and TF binding profiles with DNA methylation profiles, functional meQTLs were identified [74]. ATACseq experiments were conducted, showing the presence of chromatin accessibility peaks in CpG rich areas as well as verified CTCF binding sites in PARK16 locus by use of overlapping ChIP-seq data from ENCODE. There is recent evidence that >95% CTCF sites bound by cohesin mediate chromatin looping [75] and regulates chromatin accessibility and transcription [76]. The CTCF site has in fact been previously identified as a key area involved in long-range interactions with meQTLs rs708723 (RAB7L1) and rs960603 (PM20D1) [53]. The instant study identified rs960603 and rs708727 as meQTLs to also be associated with CPSP. In addition, Homer bioinformatics analyses showed enrichment for repressive histone marks and TF binding sites at CpG sites with significant enrichment for MAF, Zinc finger proteins, GATA and homeodomain family TF. In a case-control study of 5 PARK16 locus (RAB7L1 and SLC41A1) variants for PD, which found them to be protective, a putative GATA1 binding site was previously identified that is potentially altered by variants in the promoter region of RAB7L1 [77], consistent with the instant results showing enrichment for GATA1 binding sites at the 127 CpGs that causally mediate associations between SNPs and CPSP (FIG. 3B). In another study, the allele rs823144 A >C within the PARK16 locus was found to be protective for PD, with differential binding of particular TFs proposed to contribute to altered expression levels of the RAB7L1 gene [55]. The current findings of DNA methylation mediating association of meQTLs with phenotype (CPSP) provides a plausible putative mechanism for altered TFBS at this locus for neural phenotypes involving dopaminergic processes.

CIT was used to further examine which of the CpG sites may be causal mediators of SNP-CPSP associations. A limitation of this study is the relatively low standards when selecting putative candidate sites. Due to the exploratory nature of the study and small sample size, no priori power analysis or correction on multiple testing was conducted. In compensation, the methylation difference was taken into account. Only sites that showed at least 5% difference between groups were considered. The previous study demonstrated that this strategy could be effective and sometimes more critical in the identification of differentially methylated positions [39]. Replication studies are needed to draw any solid conclusions. While there are challenges to applying CIT methods related to low power of multiple testing; simulation studies have demonstrated the validity and advantage of the CIT package over other common multiple testing strategies [38]. The functional interpretation of the current study results are limited by the absence of experimental data from relevant cell lines and expression data, given that meQTLs are enriched in regulatory domains and are known to both enhance and repress gene expression in a cell- and tissue-specific way. Hence, it was verified using G-TEx portal that meQTLs associated with increased DNA methylation was associated with decreased gene expression for PM20D1 (eQTLs) in brain and blood tissue, and vice versa for RAB7L1 with no effect on expression of other PARK16 genes. Cellular heterogeneity presents a major confounding factor in epigenetic studies. Statistical methods (surrogate variable analysis (SVA 3.24.4)) were used to control batch effect and unknown confounders such as cell composition [16, 78, 79]. By using a study cohort with minimal preoperative pain, the current study design has the advantage of mitigating time dependent cause-effect questions that usually complicates DNA methylation-phenotype relationships in acute to chronic pain transitions.

### CONCLUSION

meQTLs analysis in blood suggest potential novel molecular mechanisms mediating genetic associations with CPSP. They also partially explain molecular function of non-protein-coding, CPSP associated genetic variants. What is not known is whether meQTL associated CpG sites are also subject to environmental effects and if so, the proportion of gene-environmental effects [80]. If the effects on DNA methylation are purely genetic, this will need to be considered in future epigenetic studies.

### FUTURE PERSPECTIVE

While genetic variation (meQTL) cannot currently be modified, DNA methylation may be a modifiable risk factor. Understanding epigenetic-mediating mechanisms opens the possibility of development of targeted therapeutic approaches to reprogram these modifications. The identification of TFBS associated with theme QTLs in previously known pain and dopamine function relevant genetic loci such as PARK16 renders new avenues to regulate their function and could spur new research into biomarkers and modifiable strategies in genetically susceptible individuals. The instant study provides novel pilot data for future transcriptome and TF binding studies to evaluate key DNA methylation sites. Future multi-omics studies of gene expression are needed to validate the current findings and identify the SNP-DNA methylation-mRNA associations in relation to CPSP and chromatin structure profiles to identify functional meQTLs [81]. Single-cell assays and programmable nucleases could be used to further explore the function of non-coding variants on DNA methylation [82], and environmental stimuli exposure could be tested to understand how meQTLs encode environmental interactions by regulating DNA methylation.

### REFERENCES

1. Macrae W A, Davies H T O. In: Epidemiology of pain. Crombie I K (Ed.). IASP Press, Seattle, 125-142 (1999).
2. Rabbitts J A, Fisher E, Rosenbloom B N, Palermo T M. Prevalence and predictors of chronic postsurgical pain in children: a systematic review and meta-analysis. J. Pain 18(6), 605-614 (2017).
3. Thapa P, Euasobhon P. Chronic postsurgical pain: current evidence for prevention and management. Korean J. Pain 31(3), 155-173 (2018).
4. Katz J, Seltzer Z. Transition from acute to chronic postsurgical pain: risk factors and protective factors. Expert Rev. Neurother. 9(5), 723-744 (2009).
5. Clarke H, Katz J, Flor H, Rietschel M, Diehl S R, Seltzer Z. Genetics of chronic post-surgical pain: a crucial step toward personal pain medicine. Can. J. Anaesth. 62(3), 294-303 (2015).
6. Chidambaran V, Gang Y, Pilipenko V, Ashton M, Ding L. Systematic review and meta-analysis of genetic risk of developing chronic postsurgical pain. J. Pain 21(1-2), 2-24 (2020)
7. Warner S C, Van Meurs J B, Schiphof D et al. Genome-wide association scan of neuropathic pain symptoms post total joint replacement highlights a variant in the protein-kinase C gene. Eur. J. Hum. Genet. 25(4), 446-451 (2017).
8. Costigan M, Belfer I, Griffin R S et al. Multiple chronic pain states are associated with a common amino acid-changing allele in KCNS1. Brain 133(9), 2519-2527 (2010).
9. Hickey O T, Nugent N F, Burke S M, Hafeez P, Mudrakouski A L, Shorten G D. Persistent pain after mastectomy with reconstruction. J. Clin. Anesth. 23(6), 482-488 (2011).
10. Montes A, Roca G, Sabate S et al. Genetic and clinical factors associated with chronic postsurgical pain after hernia repair, hysterectomy, and thoracotomy: a two-year multicenter cohort study. Anesthesiology 122(5), 1123-1141 (2015).
11. Belfer I, Dai F, Kehlet H et al. Association of functional variations in COMT and GCH1 genes with postherniotomy pain and related impairment. Pain 156(2), 273-279 (2015).
12. Kolesnikov Y, Gabovits B, Levin A et al. Chronic pain after lower abdominal surgery: do catechol-O-methyl transferase/opioid receptor mu-1 polymorphisms contribute?. Mol. Pain 9, 19 (2013).
13. Bai G, Ren K, Dubner R. Epigenetic regulation of persistent pain. Transl. Res. 165(1), 177-199 (2015).
14. Doehring A, Geisslinger G, Lotsch J. Epigenetics in pain and analgesia: an imminent research field. Eur. J. Pain 15(1), 11-16 (2011).
15. Denk F, Mcmahon S B. Chronic pain: emerging evidence for the involvement of epigenetics. Neuron 73(3), 435-444 (2012).
16. Chidambaran V, Zhang X, Geisler K et al. Enrichment of genomic pathways based on differential dna methylation associated with chronic postsurgical pain and anxiety in children: a prospective, pilot study. J. Pain 20(7), 771-785 (2019).
17. Chidambaran V, Zhang X, Martin L J et al. DNA methylation at the mu-1 opioid receptor gene (OPRM1) promoter predicts preoperative, acute, and chronic postsurgical pain after spine fusion. Pharmgenomics Pers. Med. 10, 157-168 (2017).
18. Chidambaran V, Ashton M, Martin L J, Jegga A G. Systems biology-based approaches to summarize and identify novel genes and pathways associated with acute and chronic postsurgical pain. J. Clin. Anesth. 62, 109738 (2020).
19. Crow M, Denk F, Mcmahon S B. Genes and epigenetic processes as prospective pain targets. Genome Med. 5(2), 12 (2013).
20. Smith A K, Kilaru V, Kocak M et al. Methylation quantitative trait loci (meQTLs) are consistently detected across ancestry, developmental stage, and tissue type. BMC Genomics 15, 145 (2014).
21. Do C, Shearer A, Suzuki M et al. Genetic-epigenetic interactions in cis: a major focus in the post-GWAS era. Genome Biol. 18(1), 120 (2017).
22. Hannon E, Gorrie-Stone T J, Smart M C et al. Leveraging DNA-methylation quantitative-trait loci to characterize the relationship between methylomic variation, gene expression, and complex traits. Am. J. Hum. Genet. 103(5), 654-665 (2018).
23. Banovich N E, Lan X, Mcvicker G et al. Methylation Q TLs are associated with coordinated changes in transcription factor binding, histone modifications, and gene expression levels. PLoS Genet. 10(9), e1004663 (2014).
24. Maurano M T, Humbert R, Rynes E et al. Systematic localization of common disease-associated variation in regulatory DNA. Science 337(6099), 1190-1195 (2012).
25. Hannon E, Spiers H, Viana J et al. Methylation Q TLs in the developing brain and their enrichment in schizophrenia risk loci. Nat. Neurosci. 19(1), 48-54 (2016).
26. Tang Y, Jin B, Zhou L, Lu W. MeQTL analysis of childhood obesity links epigenetics with a risk SNP rs17782313 near MC4R from meta-analysis. Oncotarget 8(2), 2800-2806 (2017).
27. Rushton M D, Reynard L N, Young D A et al. Methylation quantitative trait locus analysis of osteoarthritis links epigenetics with genetic risk. Hum. Mol. Genet. 24(25), 7432-7444 (2015).
28. Zhang H, Wang F, Kranzler H R et al. Identification of methylation quantitative trait loci (mQTLs) influencing promoter DNA methylation of alcohol dependence risk genes. Hum. Genet. 133(9), 1093-1104 (2014).
29. Lin D, Chen J, Perrone-Bizzozero N et al. Characterization of cross-tissue genetic-epigenetic effects and their patterns in schizophrenia. Genome Med. 10(1), 13 (2018).
30. Von Baeyer C L. Numerical rating scale for self-report of pain intensity in children and adolescents: recent progress and further questions. Eur. J. Pain 13(10), 1005-1007 (2009).
31. Silverman W K, Fleisig W, Rabian B, Peterson R A. Childhood anxiety sensitivity index. J. Clin. Child Psychol. 20, 162-168 (1991).
32. Chidambaran V, Ding L, Moore D L et al. Predicting the pain continuum after adolescent idiopathic scoliosis surgery: a prospective cohort study. Eur. J. Pain 21(7), 1252-1265 (2017).
33. Gerbershagen H J, Rothaug J, Kalkman C J, Meissner W. Determination of moderate-to-severe postoperative pain on the numeric rating scale: a cut-off point analysis applying four different methods. Br. J. Anaesth. 107(4), 619-626 (2011).
34. Wang K, Li M, Hakonarson H. ANNOVAR: functional annotation of genetic variants from high-throughput sequencing data. Nucleic Acids Res. 38(16), e164-e164 (2010).
35. Purcell S, Neale B, Todd-Brown K et al. PLINK: a tool set for whole-genome association and population-based linkage analyses. Am. J. Hum. Genet. 81(3), 559-575 (2007).
36. Shabalin A A. Matrix eQTL: ultra fast eQTL analysis via large matrix operations. Bioinformatics 28(10), 1353-1358 (2012).
37. Millstein J, Zhang B, Zhu J, Schadt E E. Disentangling molecular relationships with a causal inference test. BMC Genet. 10(1), 23 (2009).
38. Millstein J, Chen G K, Breton C V. cit: hypothesis testing software for mediation analysis in genomic applications. Bioinformatics 32(15), 2364-2365 (2016).
39. Zhang X, Chen X, Weirauch M T et al. Diesel exhaust and house dust mite allergen lead to common changes in the airway methylome and hydroxymethylome. Environ Epigenet 4(3), dvy020 (2018).
40. Consortium E P. An integrated encyclopedia of DNA elements in the human genome. Nature 489(7414), 57-74 (2012).
41. Chadwick L H. The NIH Roadmap Epigenomics Program data resource.

### Epigenomics 4(3), 317-324 (2012).

42. Grubert F, Zaugg J B, Kasowski M et al. Genetic control of chromatin states in humans involves local and distal chromosomal interactions. Cell 162(5), 1051-1065 (2015).
43. Harley J B, Chen X, Pujato M et al. Transcription factors operate across disease loci, with EBNA2 implicated in autoimmunity. Nat. Genet. 50(5), 699-707 (2018).
44. Heinz S, Benner C, Spann N et al. Simple combinations of lineage-determining transcription factors prime cis-regulatory elements required for macrophage and B cell identities. Mol. Cell 38(4), 576-589 (2010).
45. Weirauch M T, Yang A, Albu M et al. Determination and inference of eukaryotic transcription factor sequence specificity. Cell 158(6), 1431-1443 (2014).
46. Fragiadakis G K, Gaudilli'ere B, Ganio E A et al. Patient-specific immune states before surgery are strong correlates of surgical recovery. Anesthesiology 123(6), 1241-1255 (2015).
47. Gaudilliere B, Fragiadakis G K, Bruggner R V et al. Coordinated surgical immune signatures contain correlates of clinical recovery. Sci. Transl. Med. 6(255), 255ra131-255ra131 (2014).
48. Corces M R, Trevino A E, Hamilton E G et al. An improved ATAC-seq protocol reduces background and enables interrogation of frozen tissues. Nat. Methods 14(10), 959-962 (2017).
49. Kartashov A V, Barski A. BioWardrobe: an integrated platform for analysis of epigenomics and transcriptomics data. Genome Biol. 16, 158 (2015).
50. Langmead B, Trapnell C, Pop M, Salzberg S L. Ultrafast and memory-efficient alignment of short DNA sequences to the human genome. Genome Biol. 10(3), R25 (2009).
51. Zhang Y, Liu T, Meyer C A et al. Model-based analysis of ChIP-Seq (MACS). Genome Biol. 9(9), R137 (2008).
52. Ji H, Ehrlich L I, Seita J et al. Comprehensive methylome map of lineage commitment from haematopoietic progenitors. Nature 467(7313), 338-342 (2010).
53. Sanchez-Mut J V, Heyn H, Silva B A et al. PM20D1 is a quantitative trait locus associated with Alzheimer's disease. Nat. Med. 24(5), 598-603 (2018).
54. Pihlstrom L, Rengmark A, Bjornara K A et al. Fine mapping and resequencing of the PARK16 locus in Parkinson's disease. J. Hum. Genet. 60(7), 357-362 (2015).
55. Khaligh A, Goudarzian M, Moslem A et al. RAB7L1 promoter polymorphism and risk of Parkinson's disease; a case-control study. Neurol. Res. 39(5), 468-471 (2017).
56. Macleod D A, Rhinn H, Kuwahara T et al. RAB7L1 interacts with LRRK2 to modify intraneuronal protein sorting and Parkinson's disease risk. Neuron 77(3), 425-439 (2013).
57. Satake W, Nakabayashi Y, Mizuta 1 et al. Genome-wide association study identifies common variants at four loci as genetic risk factors for Parkinson's disease. Nat. Genet. 41(12), 1303-1307 (2009).
58. Blanchet P J, Brefel-Courbon C. Chronic pain and pain processing in Parkinson's disease. Prog. Neuropsychopharmacol. Biol. Psychiatry 87(Pt B), 200-206 (2018).
59. Serafini R A, Pryce K D, Zachariou V. The mesolimbic dopamine system in chronic pain and associated affective comorbidities. Biol. Psychiatry 87(1), 64-73 (2020).
60. Prasad R, Jho E H. A concise review of human brain methylome during aging and neurodegenerative diseases. BMB Rep. 52(10), 577-588 (2019).
61. Davies M N, Volta M, Pidsley R et al. Functional annotation of the human brain methylome identifies tissue-specific epigenetic variation across brain and blood. Genome Biol. 13(6), R43 (2012).
62. Hannon E, Lunnon K, Schalkwyk L, Mill J. Interindividual methylomic variation across blood, cortex, and cerebellum: implications for epigenetic studies of neurological and neuropsychiatric phenotypes. Epigenetics 11, 10 (2015).
63. Andrews S V, Ellis S E, Bakulski K M et al. Cross-tissue integration of genetic and epigenetic data offers insight into autism spectrum disorder. Nat. Commun. 8(1), 1011 (2017).
64. Huan T, Joehanes R, Song C et al. Genome-wide identification of DNA methylation QTLs in whole blood highlights pathways for cardiovascular disease. Nat. Commun. 10(1), 4267 (2019).
65. Nalls M A, Pankratz N, Lill C M et al. Large-scale meta-analysis of genome-wide association data identifies six new risk loci for Parkinson's disease. Nat. Genet. 46(9), 989-993 (2014).
66. Shimizu F, Katagiri T, Suzuki M et al. Cloning and chromosome assignment to 1 q32 of a human cDNA (RAB7L1) encoding a small GTP-binding protein, a member of the RAS superfamily. Cytogenet. Cell Genet. 77(3-4), 261-263 (1997).
67. Grill B, Bienvenut W V, Brown H M, Ackley B D, Quadroni M, Jin Y. C. elegans RPM-1 regulates axon termination and synaptogenesis through the Rab GEF GLO-4 and the Rab GTPase GLO-1. Neuron 55(4), 587-601 (2007).
68. Cooper A A, Gitler A D, Cashikar A et al. α-Synuclein blocks ER-golgi traffic and Rab1 rescues neuron loss in Parkinson's models. Science 313(5785), 324-328 (2006).
69. Verhoeven K, De Jonghe P, Coen K et al. Mutations in the small GTP-ase late endosomal protein RAB7 cause Charcot-Marie-Tooth type 2B neuropathy. Am. J. Hum. Genet. 72(3), 722-727 (2003).
70. Liu H, Wu C. Charcot Marie Tooth 2B peripheral sensory neuropathy: How Rab7 mutations impact NGF signaling?Int. J. Mol. Sci. 18(2), 324 (2017).
71. Huang S M, Bisogno T, Petros T J et al. Identification of a new class of molecules, the arachidonyl amino acids, and characterization of one member that inhibits pain. J. Biol. Chem. 276(46), 42639-42644 (2001).
72. Long J Z, Roche A M, Berdan C A et al. Ablation of PM20D1 reveals N-acyl amino acid control of metabolism and nociception. Proc. Natl Acad. Sci. USA 115(29), E6937-E6945 (2018).
73. Woolf C J, Costigan M. Transcriptional and posttranslational plasticity and the generation of inflammatory pain. Proc. Natl Acad. Sci. USA 96(14), 7723-7730 (1999).
74. Rushton M D, Reynard L N, Young D A et al. Methylation quantitative trait locus analysis of osteoarthritis links epigenetics with genetic risk. Hum. Mol. Genet. 24(25), 7432-7444 (2015).
75. Pugacheva E M, Kubo N, Loukinov D et al. CTCF mediates chromatin looping via N-terminal domain-dependent cohesin retention. Proc. Natl Acad. Sci. USA 117(4), 2020-2031 (2020).
76. Holwerda S, De Laat W. Chromatin loops, gene positioning, and gene expression. Front. Genet. 3, 217 (2012).
77. Gan-Or Z, Bar-Shira A, Dahary D et al. Association of sequence alterations in the putative promoter of RAB7L1 with a reduced Parkinson disease risk. Arch. Neurol. 69(1), 105-110 (2012).
78. Leek J T, Johnson W E, Parker H S, Jaffe A E, Storey J D. The sva package for removing batch effects and other unwanted variation in high-throughput experiments. Bioinformatics 28(6), 882-883 (2012).
79. Leek J T, Johnson W E, Parker H S, et al. Sva: surrogate variable analysis. Bioinformatics. 28(6), 882-883 (2012).
80. Lim I Y, Lin X, Kamani N. Implications of genotype and environment on variation in dna methylation. In: Handbook of Nutrition, Diet, and Epigenetics Patel V, Preedy V (Eds). Springer International Publishing, Chain, Switzerland, 1-20 (2017).
81. Lu Y-H, Wang B-H, Jiang F et al. Multi-omics integrative analysis identified SNP-methylation-mRNA: interaction in peripheral blood mononuclear cells. J. Cell. Mol. Med. 23(7), 4601-4610 (2019).
82. Farlik M, Sheffield N C, Nuzzo A et al. Single-cell DNA methylome sequencing and bioinformatic inference of epigenomic cell-state dynamics. Cell Rep. 10(8), 1386-1397 (2015).

### EQUIVALENTS

Those skilled in the art will recognize or be able to ascertain using no more than routine experimentation, many equivalents to the specific embodiments of the invention as described herein. Such equivalents are intended to be encompassed by the following claims.

All references cited herein are incorporated herein by reference in their entirety and for all purposes to the same extent as if each individual publication or patent or patent application was specifically and individually indicated to be incorporated by reference in its entirety for all purposes.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

1. A method for the prophylaxis or treatment of perioperative pain in a human patient in need thereof, the method comprising assaying, in vitro, a biological sample from the patient to determine the DNA methylation status of a plurality of CpG sites at the PARK16 locus of chromosome 1 and treating the patient whose DNA methylation status indicates a high risk of chronic post-surgical pain (CPSP) with a DNA methylation modifying agent.

2. A method for identifying a human patient who is susceptible to perioperative pain, the method comprising assaying, in vitro, a biological sample from the patient to determine the DNA methylation status of a plurality of CpG sites at the PARK16 locus of chromosome 1.

3. The method of claim 1, wherein the perioperative pain is selected from preoperative pain, acute postoperative pain, and chronic postoperative pain.

4. The method of claim 3, wherein the perioperative pain is chronic postoperative pain (CPSP).

5. The method of claim 1, wherein the plurality of CpG sites includes sites in at least the RAB7L1 and PM20D1 genes, and wherein the patient is identified as susceptible to CPSP where there is decreased DNA methylation at RAB7L1 CpG sites and increased DNA methylation at PM20D1 CpG sites.

6. The method of claim 1, wherein the biological sample is a whole blood sample.

7. The method of claim 6, wherein the patient identified as susceptible to CPSP is administered a therapeutic agent selected from a demethylating agent and an inhibitor of the repressor element-1 silencing transcription factor (REST).

8. The method of claim 7, wherein the agent is administered before or after a surgical procedure is performed on the patient.

9. The method of claim 7, wherein the demethylating agent is selected from procaine, zebularine and decitabine, or a combination of two or more of the foregoing.

10. The method of claim 9, wherein the demethylating agent is zebularine, decitabine, or a combination of two or more of the foregoing.

11. The method of claim 1, wherein the biological sample is assayed by a method comprising isolation of genomic DNA from the biological sample.

12. The method of claim 1, wherein the biological sample is assayed by a method comprising, or further comprising, pyrosequencing.

13. The method of claim 12, wherein the pyrosequencing comprises two or more rounds of a polymerase chain reaction.

14. The method of claim 2, wherein the perioperative pain is selected from preoperative pain, acute postoperative pain, and chronic postoperative pain.

15. The method of claim 2, wherein the perioperative pain is chronic postoperative pain (CPSP).

16. The method of claim 2, wherein the plurality of CpG sites includes sites in at least the RAB7L1 and PM20D1 genes, and wherein the patient is identified as susceptible to CPSP where there is decreased DNA methylation at RAB7L1 CpG sites and increased DNA methylation at PM20D1 CpG sites.

17. The method of claim 2, wherein the biological sample is a whole blood sample.

18. The method of claim 2, wherein the assaying comprises isolation of genomic DNA from the biological sample.

19. The method of claim 2, wherein the assaying comprises pyrosequencing.
